(19) United States
(12) Patent Application Publication
(10) Pub. No.: US 2007/0172478 A1
(43) Pub. Date: Jul. 26, 2007

(54) Methods of treating skin disorders

(75) Inventor: Daniel Magilavy, Kensington, NH (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: ASTELLAS US LLC, Deerfield, IL (US)

(21) Appl. No.: 10/588,323

(22) PCT Filed: Feb. 7, 2005
(86) PCT No.: PCT/US05/03907

§ 371 Date: Feb. 16, 2007

Related U.S. Application Data
(60) Provisional application No. 60/542,311, filed on Feb. 6, 2004.

(51) Int. Cl. A61K 39/395

(52) U.S. Cl. 424/144.1

(57) ABSTRACT

Methods of treating skin disorders are provided.

### RELATED APPLICATIONS

This application claims the benefit of U.S. Provisional Application No. 60/542,311 filed Feb. 6, 2004, the entire contents of which are hereby incorporated by reference herein.

### BACKGROUND

Psoriasis affects about 4.5 million U.S. adults. AMEVIVE® (alefacept) is a biologic agent approved for use to treat psoriasis.

### SUMMARY OF THE INVENTION

The invention provides methods of treating a variety of conditions, including T-cell mediated conditions, e.g., memory T-cell-mediated conditions, e.g., skin conditions such as psoriasis, atopic dermatitis, cutaneous T cell lymphoma, allergic and irritant contact dermatitis, lichen planus, alopecia greata, pyoderma gangrenosum, vitiligo, ocular cicatricial pemphigoid, UV damage and urticaria. The methods described herein relate to the administration of multiple cycles of an inhibitor of the LFA-3/CD2 interaction, e.g., a soluble LFA-3 polypeptide, e.g., a soluble LFA-3-immunoglobulin (Ig) fusion protein such as AMEVIVE® (alefacept) (hereinafter AMEVIVE). Multiple cycles of treatment with such agents have been found to provide more significant results (e.g., notably longer remission periods) than a single cycle of therapy or a double cycle of therapy with, surprisingly, no apparent additional risk of side effects. In a preferred embodiment, the methods described herein relate to treatment of psoriasis.

Accordingly, in one aspect, the invention features a method of treating a condition, e.g., a skin condition such as psoriasis or other skin condition described herein. In one embodiment, the condition is a mediated by memory effector T cells. The method includes administering a multiple course of treatment (preferably at least three cycles of treatment) of a soluble, CD2-binding LFA-3 polypeptide to a subject.

Preferably, the soluble, CD2-binding LFA-3 polypeptide is an LFA-3 fusion protein, e.g., an LFA-3/immunoglobulin (Ig) fusion protein. An exemplary LFA-3/Ig fusion protein includes a soluble, CD2 binding LFA-3 polypeptide fused to all or part of an Fc region of an IgG, e.g., fused to all or part of an IgG heavy chain hinge region and all or part of a heavy chain constant region. In a preferred embodiment, the Ig fusion protein consists of the amino terminal 92 amino acids of mature LFA-3, the C-terminal 10 amino acids of a human IgG1 hinge region, a CH2 region of a human IgG1 heavy chain, and all or at least part of a CH3 region of a human IgG1 heavy chain. One such fusion protein is AMEVIVE. AMEVIVE is encoded by an insert contained in plasmid pSAB152, deposited with American Type Culture Collection under the accession number ATCC 68720. AMEVIVE is described in more detail herein below.

The multiple course of treatment includes at least three cycles of treatment, each cycle including (a) an administration period during which a therapeutic agent is administered at least twice, followed by (b) a rest period during which the therapeutic agent is not administered, wherein the rest period is substantially longer than the interval between administrations (IA) in the cycle, and preferably at least as long as the administration period. In some embodiments, the multiple course includes at least four cycles, five cycles, six cycles, seven cycles, eight cycles, nine cycles, ten cycles, twelve cycles of treatment or more. The administration period of each cycle of the multiple course can be preselected or is determined by, e.g., a health care provider for the particular patient. Typically, the administration period is sufficiently long to elicit a therapeutic response, e.g., to elicit a selected level of remission as measured by a clinical measure such as a PASI score. In some embodiments, the administration period is at least 8 weeks, at least 10 weeks, at least 12 weeks, at least 14 weeks, 20 weeks or more, but is typically between 4 and 24 weeks. In a preferred embodiment, each cycle consists of 12 weeks of once-weekly administration of the polypeptide followed by 12 weeks of rest during which the patient is evaluated at least once for an effect of the agent, e.g., a therapeutic effect or a side effect. In a preferred embodiment, the rest period of each successive cycle of the multiple course is longer than the rest period of a previous cycle in the multiple course. In some embodiments, the rest period of the last cycle of the multiple course can be at least 2 years, at least 18 months, at least 3 years, 4 years, five years or longer.

The soluble, CD2 binding LFA-3 polypeptide can be administered at a dosage ranging from about 0.001 to about 50 mg binding agent per kg body weight. In one embodiment, the polypeptide is administered systemically, preferably by intramuscular (IM) or intravenous (IV) route. The administration period typically includes periodic administration of the polypeptide, e.g., once a week, twice a week, semi-weekly, or monthly. The polypeptide is typically administered at a unit dosage ranging from 2 to 15 mg when administered by IV route (for example, 7.5 mg IV bolus) and a unit dosage ranging from 2 to 30 mg when administered by IM route (for example, 10 or 15 mg IM injection).

In one embodiment, the method includes evaluating the subject for the effects of the soluble CD2-binding LFA-3 polypeptide during one or both of the administration period and the rest period of each cycle in the multiple course.

In one embodiment, the method includes administering to the subject an additional therapeutic or prophylactic agent during the multiple course of treatment, e.g., UV radiation (e.g., UVB radiation) cyclosporin A, prednisone, FK506, methotrexate, steroids, retinoids, interferon, and nitrogen mustard. The additional agent can be administered during the administration period, during the rest period, or both, during one or more cycles.

The subject is preferably a human. Preferred subjects include those who have symptoms of a T cell-mediated skin disorder such as psoriasis, e.g., dermal cell proliferation, raised red plaque formation, scalyness, itching, cracking, stinging, burning, or bleeding plaques, and those who have been diagnosed with psoriasis.

In another aspect, the invention features methods of treating a subject having psoriasis. The method includes (a) selecting a subject having had at least two cycles, e.g., selecting a subject on the basis of having had at least two cycles, of treatment with a soluble CD2-binding LFA-3 polypeptide, and (b) administering a further cycle, e.g., a third, fourth, fifth, sixth, seventh, eighth, ninth, tenth or more cycle, of treatment of a soluble CD2-binding LFA-3 polypeptide to the subject.

In another aspect, the invention features methods of treating a subject, or advising or counseling a subject to be treated, with a soluble, CD2-binding LFA-3 polypeptide described herein. The method includes instructing or providing instructions to a subject, or another individual, e.g., a health care provider, e.g., a physician, nurse, an employee of a hospital, HMO or other entity that provides health care, that the subject should be administered a multiple course of treatment described herein.

The methods described herein can be used to treat any condition mediated by memory effector T cells. The methods described herein can be used to treat skin conditions such as psoriasis and scleroderma, and non-skin conditions such as inflammatory bowel diseases, uveitis, psoriatic arthritis, rheumatoid arthritis, multiple sclerosis, and scleroderma.

### BRIEF DESCRIPTION OF THE DRAWINGS

FIGS. 1A and 1B depict the amino acid and nucleotide sequences of an LFA-3/IgG fusion protein. The signal peptide corresponds to amino acids 1-28 of FIG. 1A; the mature LFA-3 region corresponds to amino acids 29-120 of FIG. 1A; and the IgG1 region corresponds to amino acids 121-347 of FIG. 1A.

FIG. 2 is a bar graph of the percentage of psoriasis patients achieving PASI 50 at two or twelve weeks after treatment for cycles A-D of a multiple course of treatment with AMEVIVE.

FIG. 3 is a graph of the benefit and repeat response in a multiple course of treatment with AMEVIVE for psoriasis.

FIG. 4 is a bar graph of the maximum length of response time in four psoriasis patients receiving a multiple course of treatment with AMEVIVE.

FIG. 5 is a graph of the mean CD4+ T-cell counts for patients having a multiple course of treatment with AMEVIVE.

FIG. 6A is a graph of the percentage of patients achieving PASI 75 at any time during a multiple course of intravenous (IV) treatment with AMEVIVE. FIG. 6B is a graph of the percentage of patients achieving PASI 50 at any time during a multiple course of IV treatment with AMEVIVE.

FIG. 7A is a graph of the percentage of patients achieving a PGA of “clear” or “almost clear” responses at any time during a multiple course of IV treatment with AMEVIVE. FIG. 7B is a graph of the percentage of patients achieving a PGA of “clear” or “almost clear” responses at any time during a multiple course of IM treatment with AMEVIVE.

### DETAILED DESCRIPTION

The methods described herein relate generally to multiple course therapy with a soluble, CD2 binding LFA-3 polypeptide for the treatment of T cell-mediated disorders (e.g., psoriasis). Multiple course therapy has been found to provide notably longer remission periods than a single or double cycle of therapy with, surprisingly, no apparent additional risk of side effects.

Multiple Course Treatment

As used herein, a “cycle” of treatment includes (a) an administration period during which a therapeutic agent is administered at least twice (the interval between administrations is referred to as the IA), followed by (b) a rest period during which the therapeutic agent is not administered. The rest period is substantially longer, e.g., at least 4-5 times longer, than the longest IA, and is preferably at least as long as the administration period. During the administration period of the cycle, the agent can be administered at least 2, 4, 6, 8, 10, 12, 14, 16, 18 or 20 times at (preferably regular) intervals. Typically, the administration period is sufficiently long for a patient to exhibit a pre-selected level of amelioration of disease, e.g., a preselected PASI score, e.g., PASI 50 or PASI 75. The rest period can include monitoring the patient for a response to the therapeutic agent (e.g., a therapeutic effect or a side-effect). In a preferred cycle, the agent is administered once a week during a 12-week administration period followed by a 12-week rest period during which the patient is evaluated by a health care provider at least once.

In preferred embodiment there will be less than 50, 40, 30, 20 or 15 administrations during the administration period. In preferred embodiments the greatest interval between any two adjacent administrations (IA) in the administration period of the cycle is less than 30, less than 20, less than 15 or less than 10 days. In preferred embodiments the interval between administrations is about a week. The administration period of the cycle can vary with regard to dosing strategy. For example, if the administration period is measured in weeks or months, the administration period can include monthly, weekly, bi-weekly, semi-weekly, or daily administration of the agent for a specified number of weeks, as determined by a heath care practitioner for a particular patient. A preferred administration period of a cycle includes about 6-24 administrations with an IA of 3-15 days. More preferably, the administration period of a cycle includes about 10-14 administrations with an IA of 5-9 days.

In some cases, the rest period is as long as, or longer than, the period during which the agent has a substantial remittive effect on the patient, as measured by a standard clinical measure. For example, the rest period for a psoriasis patient during a cycle of treatment can be the period during which a specified Psoriasis Area and Severity Index (PASI) score (e.g., PASI 50 or PASI 75) or a specified Physician Global Assessment (PGA) score (e.g., PGA “clear” or “almost clear”) is maintained, or longer. However, a rest period that is at least as long as the remission period is typically between 1 and 10 years, e.g., between 2 and 10 years, e.g., between 2 and 5 years. In some embodiments, the rest period is at least 1 year, preferably at least 18 months, 2 years, 30 months, 36 months, 42 months, 48 months or longer.

A “multiple course of treatment” means at least three cycles of treatment. The cycles within a multiple course of treatment can be identical but they need not be identical, e.g., they may be different in dosing strategy during the administration period; or in duration of either the IA, length of administration period, rest period, or both. For example, a multiple course of treatment may include (a) a first cycle consisting of 12 weeks of once-weekly administration of AMEVIVE followed by 12 weeks of rest, followed by (b) a second cycle consisting of 12 weeks of once-weekly administration of AMEVIVE followed by a rest period of one year during which the agent has a substantial remittive effect on the patient, followed by (c) a third cycle consisting of 10 weeks of semi-weekly administration of the therapeutic agent followed by two years of rest, followed optionally by (d) successive cycles, e.g., additional fourth, fifth, sixth, seventh, eighth cycles or more.

In preferred embodiments, the rest period and the remittive effect increase with increasing number of cycles during a multiple course of treatment. The increase in the duration of the rest period or remittive effect with each increasing cycle of treatment is preferably at least 10%, more preferably at least 15% or 20%, more preferably at least 25%, 30%, 40%, 50% or more. In some embodiments, the rest period and remittive effect for the third cycle (and/or subsequent cycles) in a multiple course of treatment is at least 18 months, preferably at least 2 years, more preferably at least 30 months, 36 months, 42 months, 48 months or more.

Inhibitors of the CD2:LFA-3 Interaction

Any inhibitor of the CD2:LFA-3 interaction is useful in the methods of this invention. Such inhibitors include soluble LFA-3 polypeptides, anti-LFA-3 antibody homologs, anti-CD2 antibody homologs, soluble CD2 polypeptides, small molecules (e.g., a chemical agent having a molecular weight of less than 2500 Da, preferably, less than 1500 Da, a chemical, e.g., a small organic molecule, e.g., a product of a combinatorial library), LFA-3 and CD2 mimetic agents and derivatives thereof.

Preferred inhibitors for use in the methods described herein are soluble, CD2-binding LFA-3 polypeptides.

Soluble CD2 and LFA-3 Polypeptides

Soluble LFA-3 polypeptides or soluble CD2 polypeptides that inhibit the interaction of LFA-3 and CD2 are useful in the methods of the present invention. Soluble LFA-3 polypeptides, in particular soluble LFA-3/Ig fusions, are preferred.

As used herein, a “soluble CD2-binding LFA-3 polypeptide” is a polypeptide that includes at least the CD2-binding domain of LFA-3 (SEQ ID NO:2) and is incapable of anchoring itself in a membrane. Such soluble polypeptides include, for example, LFA-3 polypeptides that lack a sufficient portion of their membrane spanning domain to anchor the polypeptide or are modified such that the membrane spanning domain is non-functional. Soluble CD2-binding LFA-3 polypeptides include soluble fusion proteins that include at least the CD2-binding domain of LFA-3 fused to a heterologous polypeptide. In one embodiment, the heterologous polypeptide is an Fc region of an immunoglobulin (e.g., an IgG1 hinge region and CH2-CH3 domains) or a substantial portion thereof.

Soluble LFA-3 polypeptides may be derived from the transmembrane form of LFA-3, particularly the extracellular domain (e.g., amino acids 1-187 of SEQ ID NO:2 of U.S. Pat. No. 6,162,432, which is hereby incorporated by reference). Such polypeptides are described in U.S. Pat. No. 4,956,281 and U.S. Pat. No. 6,162,432, which are herein incorporated by reference. Preferred soluble LFA-3 polypeptides include polypeptides that include residues 1-92 of SEQ ID NO:2, residues 1-80 of SEQ ID NO:2, residues 50-65 of SEQ ID NO:2 and resides 20-80 of SEQ ID NO:2, wherein SEQ ID NO:2 is shown in U.S. Pat. No. 6,162,432. A vector comprising a DNA sequence (SEQ ID NO:1) encoding the amino acid sequence of SEQ ID NO:2 is deposited with the American Type Culture Collection, Rockville, Md. under Accession No. 75107, wherein SEQ ID NO:1 and 2 are shown in U.S. Pat. No. 6,162,432.

Soluble LFA-3 polypeptides may also be derived from the PI-linked form of LFA-3, such as those described in PCT Patent Application Serial No. WO 90/02181. A vector comprising a DNA sequence (SEQ ID NO:3) encoding PI-linked LFA-3 is deposited with the American Type Culture Collection, Rockville, Md. under Accession No. 68788. It is to be understood that the PI-linked form of LFA-3 and the transmembrane form of LFA-3 have identical amino acid sequences through the entire extracellular domain. Accordingly, the preferred PI-linked LFA-3 polypeptides are the same as for the transmembrane form of LFA-3.

The most preferred soluble CD2 binding LFA-3 polypeptides for use in the present invention are LFA-3/Ig fusion proteins. One example of such a fusion protein is AMEVIVE® (alefacept).

AMEVIVE® (Alefacept)

AMEVIVE is a fusion protein that includes the first extracellular domain of human LFA-3 (CD58) fused to an Fc portion of human IgG1. In particular, AMEVIVE includes the amino terminal 92 amino acids of mature LFA-3, the C-terminal 10 amino acids of a human IgG1 hinge region containing the two cysteine residues thought to participate in interchain disulfide bonding, and a substantial part of the C_{H}2 and C_{H}3 regions of a human IgG1 heavy chain constant domain (e.g., SEQ ID NO:8). The protein is a glycosylated, disulfide linked dimer with a molecular weight of about 112 kD under PAGE nonreducing conditions. The constant region of AMEVIVE has C-terminal variability that corresponds to a splice variant form of the full length fusion polypeptide.

A plasmid, pSAB152, encoding AMEVIVE is deposited with American Type Culture Collection, Rockville, Md., under the accession number ATCC 68720.

pMDR(92)Ig-3 is an example of an expression vector that can be used to produce AMEVIVE. pMDR(92)Ig-3 includes the following elements: (a) A segment of pBR322 containing the ColE1 origin and beta lactamase expression cassette (GenBank Accession No. J01749); (b) DHFR expression cassette consisting of: SV40 early promoter with the enhancer deleted (a portion of GenBank Accession No. J02400), murine DHFR cDNA (GenBank Accession No. L26316), SV40 poly A site and small t intron (portions of GenBank Accession No. J02400), and human gastrin transcription terminator sequence, 3′UTR (Sato et al. (1986) Mol Cell Biol 6:1032-1043); (c) an AMEVIVE expression cassette including, preferably in the following order: The SV40 early promoter/enhancer (GenBank Accession No. J02400), Adenovirus Major Late Promoter and tripartite leader, including a splice donor and intron sequence (a portion of GenBank Accession No. J01917), murine Ig heavy chain variable region intron sequence and splice acceptor (Kaufman and Sharp (1982) Mol Cell Biol. 2: 1304-1319, (optionally) cloning linkers, the first 92 amino acids of LFA-3 gene as isolated from a human tonsil cDNA library, fused in frame to a nucleic acid encoding the hinge CH2 and CH3 regions of a human IgG1 gene as isolated from a human fibroblastic genomic DNA library, cloning linkers (optionally), MIS 3′ UT region including poly A site (GenBank Accession No. K03474), and SV40 polyA site and small t intron (GenBank Accession No. J02400); and a segment of pBR327 (GenBank Accession No. L08856).

Host cell lines that can be used to produce AMEVIVE can be derived from CHO-DUkX-B1 cells. In one embodiment, a DHFR(−) mutant of this cell line can be transfected with the vector pMDR(92)Ig-3, and DHFR(+) transformants can be cultured in selective medium (e.g., containing 25 nM of methotrexate (MTX)). Positive transformants can be subjected to increasing concentrations of MTX (e.g., 50 nM), and colonies producing high levels of AMEVIVE can then be selected.

Production of AMEVIVE can be carried out as follows: CHO host cells are thawed, scaled up to a culture of 2000 L, maintained in culture for 6-7 days with pH control and nutrient feed (at 48 hrs., 96 hrs., and 120 hrs.), after which conditioned medium is harvested through microfiltration. MTX is preferably present in the culture medium. AMEVIVE can be recovered from the conditioned medium by carrying out the following steps: (i) Protein A chromatography, (ii) ceramic hydroxyapatite chromatography, (iii) viral inactivation at low pH, (iv) hydrophobic interaction chromatography, (v) followed by concentration, diafiltration, viral filtration, and a second concentration step which yields fusion product.

Another way of producing AMEVIVE for use in the methods of this invention is described in co-pending, commonly assigned U.S. patent application Ser. No. 07/770,967. Generally, conditioned culture medium of COS7 or CHO cells transfected with pSAB152 was concentrated using an AMICON S1Y30 spiral cartridge system (AMICON, Danvers, Mass.) and subjected to Protein A-Sepharose 4B (Sigma, St. Louis, Mo.) chromatography. The bound proteins were eluted and subjected to Superose-12 (Pharmacia/LKB, Piscataway, N.J.) gel filtration chromatography.

Superose-12 fractions containing AMEVIVE with the least amount of contaminating proteins, as determined on SDS-PAGE gels and by Western blot analysis, (see, e.g., Towbin et al., Proc. Natl. Acad. Sci. USA, 74, pp. 4350-54 (1979); Antibodies: A Laboratory Manual, pp. 474-510 (Cold Spring Harbor Laboratory (1988)), were pooled and concentrated in a YM30 Centricon (AMICON). AMEVIVE was detected on Western blots using a rabbit anti-LFA-3 polyclonal antiserum, followed by detectably labeled goat anti-rabbit IgG. The purified AMEVIVE of COS7 or CHO cells was a dimer of two monomeric LFA-3-Ig fusion proteins, connected by disulfide bonds.

LFA-3-Ig fusion activity can be tested using the following bioassays: (1) a CD32/64 (Fc gamma R1/R1) U937 cell bridging assay, and (2) a CD16 (Fc gamma RIII) Jurkat cell bridging assay. Both assays test the ability of AMEVIVE to bridge CHO cells displaying cell surface CD2 to cells expressing Fc-gamma receptors. The latter assay, assay (2), involves culturing adherent CH0—CD2 cells to form a monolayer in 96-well plates; adding AMEVIVE controls and samples; adding fluorescently labeled Jurkat-CD16(+); and measuring fluorescence intensity.

Binding of LFA-3-Ig fusion to CD2 immobilized onto a substrate, e.g., a chip, can also be used to test the fusion proteins.

CD2 Polypeptides

Soluble CD2 polypeptides may be derived from full length CD2, particularly the extracellular domain. Such polypeptides may comprise all or part of the extracellular domain of CD2. Exemplary soluble CD2 polypeptides are described in PCT WO 90/08187, which is herein incorporated by reference.

Production of Soluble Polypeptides

The production of the soluble polypeptides useful in this invention may be achieved by a variety of methods known in the art. For example, the polypeptides may be derived from intact transmembrane LFA-3 or CD2 molecules or an intact PI-linked LFA-3 molecule by proteolysis using specific endopeptidases in combination with exopeptidases, Edman degradation, or both. The intact LFA-3 molecule or the intact CD2 molecule, in turn, may be purified from its natural source using conventional methods. Alternatively, the intact LFA-3 or CD2 may be produced by known recombinant DNA techniques using cDNAs (see, e.g., U.S. Pat. No. 4,956,281 to Wallner et al.; Aruffo and Seed, Proc. Natl. Acad. Sci., 84, pp. 2941-45 (1987); Sayre et al., Proc. Natl. Acad. Sci. USA, 84, pp. 2941-45 (1987)).

Preferably, the soluble polypeptides useful in the present invention are produced directly, thus eliminating the need for an entire LFA-3 molecule or an entire CD2 molecule as a starting material. This may be achieved by conventional chemical synthesis techniques or by well-known recombinant DNA techniques wherein only those DNA sequences that encode the desired peptides are expressed in transformed hosts. For example, a gene that encodes the desired soluble LFA-3 polypeptide or soluble CD2 polypeptide may be synthesized by chemical means using an oligonucleotide synthesizer. Such oligonucleotides are designed based on the amino acid sequence of the desired soluble LFA-3 polypeptide or soluble CD2 polypeptide. Specific DNA sequences coding for the desired peptide also can be derived from the full length DNA sequence by isolation of specific restriction endonuclease fragments or by PCR synthesis of the specified region.

Standard methods may be applied to synthesize a gene encoding a soluble LFA-3 polypeptide or a soluble CD2 polypeptide that is useful in this invention. For example, the complete amino acid sequence may be used to construct a back-translated gene. A DNA oligomer containing a nucleotide sequence coding for a soluble LFA-3 polypeptide or a soluble CD2 polypeptide useful in this invention may be synthesized in a single step. Alternatively, several smaller oligonucleotides coding for portions of the desired polypeptide may be synthesized and then ligated. Preferably, a soluble LFA-3 polypeptide or a soluble CD2 polypeptide useful in this invention will be synthesized as several separate oligonucleotides that are subsequently linked together. The individual oligonucleotides typically contain 5′ or 3′ overhangs for complementary assembly.

Once assembled, preferred genes will be characterized by sequences that are recognized by restriction endonucleases (including unique restriction sites for direct assembly into a cloning or an expression vector), preferred codons taking into consideration the host expression system to be used, and a sequence that, when transcribed, produces a stable, efficiently translated mRNA. Proper assembly may be confirmed by nucleotide sequencing, restriction mapping, and expression of a biologically active polypeptide in a suitable host.

It will be appreciated by those of skill in the art that, due to the degeneracy of the genetic code, DNA molecules comprising many other nucleotide sequences will also be capable of encoding the soluble LFA-3 and CD2 polypeptides encoded by the specific DNA sequences described above. These degenerate sequences also code for polypeptides that are useful in this invention.

The DNA sequences may be expressed in unicellular hosts, or preferably in isolated mammalian host cells. As is well known in the art, in order to obtain high expression levels of a transfected gene in a host, the gene must be operatively linked to transcriptional and translational expression control sequences that are functional in the chosen expression host. Preferably, the expression control sequences, and the gene of interest, will be contained in an expression vector that further comprises a bacterial selection marker and origin of replication. If the expression host is a eukaryotic cell, the expression vector can further comprise an additional expression marker useful in the expression host.

The DNA sequences encoding the desired soluble polypeptides may or may not encode a signal sequence. If the expression host is prokaryotic, it generally is preferred that the DNA sequence not encode a signal sequence. If the expression host is eukaryotic, it generally is preferred that a signal sequence be encoded.

An amino terminal methionine may or may not be present on the expressed product. If the terminal methionine is not cleaved by the expression host, it may, if desired, be chemically removed by standard techniques. A wide variety of expression host/vector combinations may be employed.

Useful expression vectors for eukaryotic hosts, include, for example, vectors comprising expression control sequences from SV40, bovine papilloma virus, adenovirus and cytomegalovirus. Useful expression vectors for bacterial hosts include known bacterial plasmids, such as plasmids from E. coli, including col E1, pCR1, pBR322, pMB9 and their derivatives, wider host range plasmids, such as RP4, phage DNAs, e.g., the numerous derivatives of phage lambda, e.g., NM989, and other DNA phages, such as M13 and filamentous single stranded DNA phages. Useful expression vectors for yeast cells include the 2μ plasmid and derivatives thereof. Useful vectors for insect cells include pVL 941.

In addition, any of a wide variety of expression control sequences may be used in these vectors. Such useful expression control sequences include the expression control sequences associated with structural genes of the foregoing expression vectors. Examples of useful expression control sequences include, for example, the early and late promoters of SV40 or adenovirus, the lac system, the tip system, the TAC or TRC system, the major operator and promoter regions of phage lambda, the control regions of fd coat protein, the promoter for 3-phosphoglycerate kinase or other glycolytic enzymes, the promoters of acid phosphatase, e.g., Pho5, the promoters of the yeast α-mating system and other sequences known to control the expression of genes of prokaryotic or eukaryotic cells or their viruses, and various combinations thereof.

A wide variety of host cells are useful. Host cells can be a unicellular organism, or can be obtained from a multicellular organism, e.g., isolated cells from a multicellular host. These hosts may include well known eukaryotic and prokaryotic hosts, such as strains of E. coli, Pseudomonas, Bacillus, Streptomyces, fungi, yeast, insect cells such as Spodoptera frugiperda (SF9), animal cells such as CHO and mouse cells, African green monkey cells such as COS 1, COS 7, BSC 1, BSC 40, and BMT 10, and human cells, as well as plant cells in tissue culture. For animal cell expression, CHO cells and COS 7 cells are preferred.

It should be understood that not all vectors and expression control sequences will function equally well to express the DNA sequences described herein. Neither will all hosts function equally well with the same expression system. However, one of skill in the art may make a selection among these vectors, expression control sequences and hosts without undue experimentation. For example, in selecting a vector, the host must be considered because the vector must replicate in it. The vector's copy number, the ability to control that copy number, and the expression of any other proteins encoded by the vector, such as antibiotic markers, should also be considered.

In selecting an expression control sequence, a variety of factors should also be considered. These include, for example, the relative strength of the sequence, its controllability, and its compatibility with the DNA sequences discussed herein, particularly as regards potential secondary structures. Unicellular hosts should be selected by consideration of their compatibility with the chosen vector, the toxicity of the product coded for by the DNA sequences, their secretion characteristics, their ability to fold the soluble polypeptides correctly, their fermentation or culture requirements, and the ease of purification of the products coded for by the DNA sequences.

Within these parameters, one of skill in the art may select various vector/expression control sequence/host combinations that will express the desired DNA sequences on fermentation or in large scale animal culture, for example with CHO cells or COS 7 cells.

The soluble LFA-3 and CD2 polypeptides may be isolated from the fermentation or cell culture and purified using any of a variety of conventional methods. One of skill in the art may select the most appropriate isolation and purification techniques.

While recombinant DNA techniques are the preferred method of producing useful soluble CD2 polypeptides or soluble LFA-3 polypeptides having a sequence of more than 20 amino acids, shorter CD2 or LFA-3 polypeptides having less than about amino acids are preferably produced by conventional chemical synthesis techniques. Synthetically produced polypeptides useful in this invention can advantageously be produced in extremely high yields and can be easily purified.

Preferably, such soluble CD2 polypeptides or soluble LFA-3 polypeptides are synthesized by solution phase or solid phase polypeptide synthesis and, optionally, digested with carboxypeptidase (to remove C-terminal amino acids) or degraded by manual Edman degradation (to remove N-terminal amino acids). The use of solution phase synthesis advantageously allows for the direct addition of certain derivatized amino acids to the growing polypeptide chain, such as the O-sulfate ester of tyrosine. This obviates the need for a subsequent derivatization step to modify any residue of the polypeptides useful in this invention.

Proper folding of the polypeptides may be achieved under oxidative conditions that favor disulfide bridge formation as described by Kent, “Chemical Synthesis of Polypeptides and Proteins”, Ann. Rev. Biochem., 57, pp. 957-89 (1988). Polypeptides produced in this way may then be purified by separation techniques widely known in the art.

Anti-LFA-3 and Anti-CD2 Antibody Homologs

As used herein, an “antibody homolog” is a protein comprising one or more polypeptides selected from immunoglobulin light chains, immunoglobulin heavy chains and antigen-binding fragments thereof which are capable of binding to an antigen. The component polypeptides of an antibody homolog composed of more than one polypeptide may optionally be disulfide-bound or otherwise covalently crosslinked. Accordingly, antibody homologs include intact immunoglobulins of types IgA, IgG, IgE, IgD, IgM (as well as subtypes thereof), wherein the light chains of the immunoglobulin may be of types kappa or lambda. Antibody homologs also include portions of intact immunoglobulins that retain antigen-binding specificity, for example, Fab fragments, Fab′ fragments, F(ab′)₂ fragments, F(v) fragments, heavy chain monomers or dimers, light chain monomers or dimers, dimers consisting of one heavy and one light chain, and the like. The term includes recombinant antibodies, chimeric, CDR-grafted and humanized antibodies, or other antibodies modified to be less immunogenic in a human.

As used herein, a “humanized recombinant or humanized antibody homolog” is an antibody homolog, produced by recombinant DNA technology, in which some or all of the amino acids of a human immunoglobulin light or heavy chain that are required for antigen binding have been substituted for the corresponding amino acids from a nonhuman mammalian immunoglobulin light or heavy chain.

As used herein, a “chimeric recombinant antibody homolog” is an antibody homolog, produced by recombinant DNA technology, in which all or part of the hinge and constant regions of an immunoglobulin light chain, heavy chain, or both, have been substituted for the corresponding regions from another immunoglobulin light chain or heavy chain.

Many types of anti-LFA-3 or anti-CD2 antibody homologs are useful in the methods of this invention. These include monoclonal antibodies, recombinant antibodies, chimeric recombinant antibodies, humanized recombinant antibodies, as well as antigen-binding portions of the foregoing.

Among the anti-LFA-3 antibody homologs, it is preferable to use monoclonal anti-LFA-3 antibodies. It is more preferable to use a monoclonal anti-LFA-3 antibody produced by a hybridoma selected from the group of hybridomas having Accession Nos. ATCC HB 10693 (1E6), ATCC HB 10694 (HC-1B11), ATCC HB 10695 (7A6), and ATCC HB 10696 (8B8), or the monoclonal antibody known as TS2/9 (Sanchez-Madrid et al., “Three Distinct Antigens Associated with Human T-Lymphocyte-Mediated Cytolysis: LFA-1, LFA-2 and LFA-3”, Proc. Natl. Acad. Sci. USA, 79, pp. 7489-93 (1982)). Most preferably, the monoclonal anti-LFA-3 antibody is produced by a hybridoma selected from the group of hybridomas having Accession Nos. ATCC HB 10695 (7A6) and ATCC HB 10693 (1E6).

Among the anti-CD2 antibody homologs, it is preferable to use monoclonal anti-CD2 antibodies, such as the anti-CD2 monoclonal antibodies known as the T11₁ epitope antibodies, including TS2/18 (Sanchez-Madrid et al., “Three Distinct Antigens Associated with Human T-Lymphocyte-Mediated Cytolysis: LFA-1, LFA-2 and LFA-3”, Proc. Natl. Acad. Sci. USA, 79, pp. 7489-93 (1982)).

The technology for producing monoclonal antibodies is well known. See generally, Harlow, E. and Lane, D. (1988) Antibodies: A Laboratory Manual, Cold Spring Harbor Laboratory Press, Cold Spring Harbor, N.Y.; Kohler et al., Nature, “Continuous Cultures of Fused Cells Secreting Antibody of Predefined Specificity”, 256, pp. 495-97 (1975). Useful immunogens for the purpose of this invention include CD2- or LFA-3-bearing cells, as well as cell free preparations containing LFA-3, CD2 or counter receptor-binding fragments thereof (e.g., CD2 fragments that bind to LFA-3 or LFA-3 fragments that bind to CD2).

Immunization may be accomplished using standard procedures. The unit dose and immunization regimen depend on the species of mammal immunized, its immune status, the body weight of the mammal, etc. Typically, the immunized mammals are bled and the serum from each blood sample is assayed for particular antibodies using appropriate screening assays. For example, useful anti-LFA-3 or anti-CD2 antibodies may be identified by testing the ability of the immune serum to block sheep red blood cell rosetting of Jurkat cells, which results from the presence of LFA-3 and CD2 on the respective surfaces of these cells. The lymphocytes used in the production of hybridoma cells typically are isolated from immunized mammals whose sera have already tested positive for the presence of the desired antibodies using such screening assays.

Anti-CD2 and anti-LFA-3 antibody homologs useful in the present invention may also be recombinant antibodies produced by host cells transformed with DNA encoding immunoglobulin light and heavy chains of a desired antibody. Recombinant antibodies may be produced by well known genetic engineering techniques. See, e.g., U.S. Pat. No. 4,816,397, which is incorporated herein by reference. For example, recombinant antibodies may be produced by cloning cDNA or genomic DNA encoding the immunoglobulin light and heavy chains of the desired antibody from a hybridoma cell that produces an antibody homolog useful in this invention. The cDNA or genomic DNA encoding those polypeptides is then inserted into expression vectors so that both genes are operatively linked to their own transcriptional and translational expression control sequences. The expression vector and expression control sequences are chosen to be compatible with the expression host cell used. Typically, both genes are inserted into the same expression vector.

Prokaryotic or eukaryotic host cells may be used. Expression in eukaryotic host cells is preferred because such cells are more likely than prokaryotic cells to assemble and secrete a properly folded and immunologically active antibody. It is possible that the host cells will produce portions of intact antibodies, such as light chain dimers or heavy chain dimers, which also are antibody homologs according to the present invention.

It will be understood that variations on the above procedure are useful in the present invention. For example, it may be desired to transform a host cell with DNA encoding either the light chain or the heavy chain (but not both) of an antibody homolog. Recombinant DNA technology may also be used to remove some or all of the DNA encoding either or both of the light and heavy chains that is not necessary for CD2 or LFA-3 counter receptor binding. The molecules expressed from such truncated DNA molecules are useful in the methods of this invention. In addition, bifunctional antibodies may be produced in which one heavy and one light chain are anti-CD2 or anti-LFA-3 antibody homologs and the other heavy and light chain are specific for an antigen other than CD2 or LFA-3, or another epitope of CD2 or LFA-3.

Chimeric recombinant anti-LFA-3 or anti-CD2 antibody homologs may be produced by transforming a host cell with a suitable expression vector comprising DNA encoding the desired immunoglobulin light and heavy chains in which all or some of the DNA encoding the hinge and constant regions of the heavy and/or the light chain have been substituted with DNA from the corresponding region of an immunoglobulin light or heavy chain of a different species. When the original recombinant antibody is nonhuman, and the inhibitor is to be administered to a human, substitution of corresponding human sequences is preferred. An exemplary chimeric recombinant antibody has mouse variable regions and human hinge and constant regions. See generally, U.S. Pat. No. 4,816,397; Morrison et al., “Chimeric Human Antibody Molecules: Mouse Antigen-Binding Domains With Human Constant Region Domains”, Proc. Natl. Acad. Sci. USA, 81, pp. 6851-55 (1984); Robinson et al., International Patent Publication PCT/US86/02269; Akira, et al., European Patent Application 184,187; Taniguchi, M., European Patent Application 171,496; Neuberger et al., International Application WO 86/01533; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J Natl Cancer Inst. 80:1553-1559.

Humanized recombinant anti-LFA-3 or anti-CD2 antibodies can be generated by replacing sequences of the Fv variable region that are not directly involved in antigen binding with equivalent sequences from human Fv variable regions. General methods for generating humanized antibodies are provided by Morrison, S. L., 1985, Science 229:1202-1207, by Oi et al., 1986, BioTechniques 4:214, and by Queen et al. U.S. Pat. No. 5,585,089, U.S. Pat. No. 5,693,761 and U.S. Pat. No. 5,693,762, the contents of all of which are hereby incorporated by reference. Those methods include isolating, manipulating, and expressing the nucleic acid sequences that encode all or part of immunoglobulin Fv variable regions from at least one of a heavy or light chain. Sources of such nucleic acids are well known to those skilled in the art and, for example, may be obtained from a hybridoma producing an anti-LFA-3 or anti-CD2 antibody. Nucleic acids encoding the humanized antibody, or fragment thereof, can then be cloned into an appropriate expression vector.

Humanized or CDR-grafted antibody molecules or immunoglobulins can be produced by CDR-grafting or CDR substitution, wherein one, two, or all CDR's of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method that may be used to prepare the humanized antibodies of the present invention (UK Patent Application GB 2188638A, filed on Mar. 26, 1987; Winter U.S. Pat. No. 5,225,539), the contents of which are expressly incorporated by reference. All of the CDR's of a particular human antibody may be replaced with at least a portion of a non-human CDR or only some of the CDR's may be replaced with non-human CDR's. It is only necessary to replace the number of CDR's required for binding of the humanized antibody to a predetermined antigen, e.g., LFA-3 or CD2.

Also within the scope of the invention are humanized antibodies, including immunoglobulins, in which specific amino acids have been substituted, deleted or added. In particular, preferred humanized antibodies have amino acid substitutions in the framework region, such as to improve binding to the antigen. For example, a selected, small number of acceptor framework residues of the humanized immunoglobulin chain can be replaced by the corresponding donor amino acids. Preferred locations of the substitutions include amino acid residues adjacent to the CDR, or which are capable of interacting with a CDR (see e.g., U.S. Pat. No. 5,585,089). Criteria for selecting amino acids from the donor are described in U.S. Pat. No. 5,585,089, e.g., columns 12-16 of U.S. Pat. No. 5,585,089, the contents of which are hereby incorporated by reference. Other techniques for humanizing immunoglobulin chains, including antibodies, are described in Padlan et al. EP 519596 A1, published on Dec. 23, 1992.

Human monoclonal antibodies (mAbs) directed against human LFA-3 or CD2 can be generated using transgenic mice carrying the complete human immune system rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906; Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

Monoclonal antibodies can also be generated by other methods known to those skilled in the art of recombinant DNA technology. An alternative method, referred to as the “combinatorial antibody display” method, has been developed to identify and isolate antibody fragments having a particular antigen specificity, and can be utilized to produce monoclonal antibodies (for descriptions of combinatorial antibody display see, e.g., Sastry et al. 1989 PNAS 86:5728; Huse et al. 1989 Science 246:1275; and Orlandi et al. 1989 PNAS 86:3833). After immunizing an animal with an immunogen as described above, the antibody repertoire of the resulting B-cell pool is cloned. Methods are generally known for obtaining the DNA sequence of the variable regions of a diverse population of immunoglobulin molecules by using a mixture of oligomer primers and PCR (Larrick et al., 1991, Biotechniques 11:152-156; Larrick et al., 1991, Methods: Companion to Methods in Enzymology 2:106-110).

Examples of methods and reagents particularly amenable for use in generating a variegated antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J. 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982. Kits for generating phage display libraries are commercially available (e.g., the Pharmacia Recombinant Phage Antibody System, catalog no. 27-9400-01; and the Stratagene SurfZAP™ phage display kit, catalog no. 240612).

In certain embodiments, the V region domains of heavy and light chains can be expressed on the same polypeptide, joined by a flexible linker to form a single-chain Fv fragment, and the scFV gene subsequently cloned into the desired expression vector or phage genome. As generally described in McCafferty et al., Nature (1990) 348:552-554, complete VH and VL domains of an antibody, joined by a flexible (Gly4-Ser)₃ linker can be used to produce a single chain antibody which can render the display package separable based on antigen affinity. Isolated scFV antibodies immunoreactive with the antigen can subsequently be formulated into a pharmaceutical preparation for use in the subject method.

Specific antibodies with high affinities for a surface protein can be made according to methods known to those in the art, e.g., methods involving screening of libraries (Ladner, R. C., et al., U.S. Pat. No. 5,233,409; Ladner, R. C., et al., U.S. Pat. No. 5,403,484). Further, the methods of these libraries can be used in screens to obtain binding determinants that are mimetics of the structural determinants of antibodies. See for example Bajorath, J. and S. Sheriff, 1996, Proteins: Struct., Funct., and Genet. 24 (2), 152-157; Webster, D. M. and A. R. Rees, 1995, “Molecular modeling of antibody-combining sites,” in S. Paul, Ed., Methods in Molecular Biol. 51, Antibody Engineering Protocols, Humana Press, Totowa, N.J., pp 17-49; and Johnson, G., Wu, T. T. and E. A. Kabat, 1995, “Seqhunt: A program to screen aligned nucleotide and amino acid sequences,” in Methods in Molecular Biol. 51, op. cit., pp 1-15.

Anti-CD2 and anti-LFA-3 antibody homologs that are not intact antibodies are also useful in this invention. Such homologs may be derived from any of the antibody homologs described above. For example, antigen-binding fragments, as well as full-length monomeric, dimeric or trimeric polypeptides derived from the above-described antibodies are themselves useful. Useful antibody homologs of this type include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. These antibody fragments are obtained using conventional techniques known to those with skill in the art, and the fragments are screened for utility in the same manner as are intact antibodies. Anti-LFA-3 heavy chains are preferred anti-LFA-3 antibody fragments.

Antibody fragments may also be produced by chemical methods, e.g., by cleaving an intact antibody with a protease, such as pepsin or papain, and optionally treating the cleaved product with a reducing agent. Alternatively, useful fragments may be produced by using host cells transformed with truncated heavy and/or light chain genes. Heavy and light chain monomers may be produced by treating an intact antibody with a reducing agent, such as dithiothreitol, followed by purification to separate the chains. Heavy and light chain monomers may also be produced by host cells transformed with DNA encoding either the desired heavy chain or light chain, but not both. See, e.g., Ward et al., “Binding Activities of a Repertoire of Single Immunoglobulin Variable Domains Secreted from Escherichia coli”, Nature, 341, pp. 544-46 (1989); Sastry et al., “Cloning of the Immunological Repertoire in Escherichia coli for Generation of Monoclonal Catalytic Antibodies: Construction of a Heavy Chain Variable Region-Specific cDNA Library”, Proc. Natl. Acad. Sci. USA, 86, pp. 5728-32 (1989).

LFA-3 And CD-2 Mimetic or Small Molecule Agents

Also useful in the methods of this invention are LFA-3 and CD2 mimetic agents. These agents, which may be peptides, semi-peptidic compounds or non-peptidic compounds (e.g., small organic molecules), are inhibitors of the CD2:LFA-3 interaction. Preferred CD2 and LFA-3 mimetic agents will inhibit the CD2:LFA-3 interaction at least as well as anti-LFA-3 monoclonal antibody 7A6 or anti-CD2 monoclonal antibody TS2/18 (described supra).

In preferred embodiments, the test agent is a member of a combinatorial library, e.g., a peptide or organic combinatorial library, or a natural product library. In a preferred embodiment, the plurality of test compounds, e.g., library members, includes at least 10, 10², 10³, 10⁴, 10⁵, 10⁶, 10⁷, or 10⁸ compounds. In a preferred embodiment, the plurality of test compounds, e.g., library members, share a structural or functional characteristic.

In one embodiment, the invention provides libraries of LFA-3 and/or CD2 inhibitors The synthesis of combinatorial libraries is well known in the art and has been reviewed (see, e.g., E. M. Gordon et al., J. Med. Chem. (1994) 37:1385-1401 DeWitt, S. H.; Czarnik, A. W. Acc. Chem. Res. (1996) 29:114; Armstrong, R. W.; Combs, A. P.; Tempest, P. A.; Brown, S. D.; Keating, T. A. Acc. Chem. Res. (1996) 29:123; Ellman, J. A. Acc. Chem. Res. (1996) 29:132; Gordon, E. M.; Gallop, M. A.; Patel, D. V. Acc. Chem. Res. (1996) 29:144; Lowe, G. Chem. Soc. Rev. (1995) 309, Blondelle et al. Trends Anal. Chem. (1995) 14:83; Chen et al. J. Am. Chem. Soc. (1994) 116:2661; U.S. Pat. Nos. 5,359,115, 5,362,899, and 5,288,514; PCT Publication Nos. WO92/10092, WO93/09668, WO91/07087, WO93/20242, WO94/08051).

Libraries of compounds of the invention can be prepared according to a variety of methods, some of which are known in the art. For example, a “split-pool” strategy can be implemented in the following way: beads of a functionalized polymeric support are placed in a plurality of reaction vessels; a variety of polymeric supports suitable for solid-phase peptide synthesis are known, and some are commercially available (see, e.g., M. Bodansky “Principles of Peptide Synthesis”, 2nd edition, Springer-Verlag, Berlin (1993)). To each aliquot of beads is added a solution of a different activated amino acid, and the reactions are allow to proceed to yield a plurality of immobilized amino acids, one in each reaction vessel. The aliquots of derivatized beads are then washed, “pooled” (i.e., recombined), and the pool of beads is again divided, with each aliquot being placed in a separate reaction vessel. Another activated amino acid is then added to each aliquot of beads. The cycle of synthesis is repeated until a desired peptide length is obtained. The amino acid residues added at each synthesis cycle can be randomly selected; alternatively, amino acids can be selected to provide a “biased” library, e.g., a library in which certain portions of the inhibitor are selected non-randomly, e.g., to provide an inhibitor having known structural similarity or homology to a known peptide capable of interacting with an antibody, e.g., the an anti-idiotypic antibody antigen binding site. It will be appreciated that a wide variety of peptidic, peptidomimetic, or non-peptidic compounds can be readily generated in this way.

The “split-pool” strategy results in a library of peptides, e.g., inhibitors, which can be used to prepare a library of test compounds of the invention. In another illustrative synthesis, a “diversomer library” is created by the method of Hobbs DeWitt et al. (Proc. Natl. Acad. Sci. U.S.A. 90:6909 (1993)). Other synthesis methods, including the “tea-bag” technique of Houghten (see, e.g., Houghten et al., Nature 354:84-86 (1991)) can also be used to synthesize libraries of compounds according to the subject invention.

Libraries of compounds can be screened to determine whether any members of the library have a desired activity, and, if so, to identify the active species. Methods of screening combinatorial libraries have been described (see, e.g., Gordon et al., J. Med. Chem., supra). Soluble compound libraries can be screened by affinity chromatography with an appropriate receptor to isolate ligands for the receptor, followed by identification of the isolated ligands by conventional techniques (e.g., mass spectrometry, NMR, and the like). Immobilized compounds can be screened by contacting the compounds with a soluble receptor; preferably, the soluble receptor is conjugated to a label (e.g., fluorophores, colorimetric enzymes, radioisotopes, luminescent compounds, and the like) that can be detected to indicate ligand binding. Alternatively, immobilized compounds can be selectively released and allowed to diffuse through a membrane to interact with a receptor. Exemplary assays useful for screening the libraries of the invention are described below.

In one embodiment, compounds of the invention can be screened for the ability to interact with a CD2 or LFA-3 polypeptide by assaying the activity of each compound to bind directly to the polypeptide or to inhibit a CD2:LFA-3 interaction, e.g., by incubating the test compound with a CD2 or LFA-3 polypeptide and a lysate, e.g., a T or APC cell lysate, e.g., in one well of a multiwell plate, such as a standard 96-well microtiter plate. In this embodiment, the activity of each individual compound can be determined. A well or wells having no test compound can be used as a control. After incubation, the activity of each test compound can be determined by assaying each well. Thus, the activities of a plurality of test compounds can be determined in parallel.

In still another embodiment, large numbers of test compounds can be simultaneously tested for binding activity. For example, test compounds can be synthesized on solid resin beads in a “one bead-one compound” synthesis; the compounds can be immobilized on the resin support through a photolabile linker. A plurality of beads (e.g., as many as 100,000 beads or more) can then be combined with yeast cells and sprayed into a plurality of “nano-droplets”, in which each droplet includes a single bead (and, therefore, a single test compound). Exposure of the nano-droplets to UV light then results in cleavage of the compounds from the beads. It will be appreciated that this assay format allows the screening of large libraries of test compounds in a rapid format.

Combinatorial libraries of compounds can be synthesized with “tags” to encode the identity of each member of the library (see, e.g., W. C. Still et al., U.S. Pat. No. 5,565,324 and PCT Publication Nos. WO 94/08051 and WO 95/28640). In general, this method features the use of inert, but readily detectable, tags that are attached to the solid support or to the compounds. When an active compound is detected (e.g., by one of the techniques described above), the identity of the compound is determined by identification of the unique accompanying tag. This tagging method permits the synthesis of large libraries of compounds that can be identified at very low levels. Such a tagging scheme can be useful, e.g., in the “nano-droplet” screening assay described above, to identify compounds released from the beads.

In preferred embodiments, the libraries of compounds of the invention contain at least 30 compounds, more preferably at least 100 compounds, and still more preferably at least 500 compounds. In preferred embodiments, the libraries of compounds of the invention contain fewer than 10⁹ compounds, more preferably fewer than 10⁸ compounds, and still more preferably fewer than 10⁷ compounds.

Derivatized Inhibitors

Also useful in the methods of this invention are derivatized inhibitors of the CD2:LFA-3 interaction in which, for example, any of the antibody homologs, soluble CD2 and LFA-3 polypeptides, or CD2 and LFA-3 mimetic agents described herein are functionally linked (by chemical coupling, genetic fusion or otherwise) to one or more members independently selected from the group consisting of anti-LFA-3 and anti-CD2 antibody homologs, soluble LFA-3 and CD2 polypeptides, CD2 and LFA-3 mimetic agents, cytotoxic agents and pharmaceutical agents.

One type of derivatized inhibitor is produced by crosslinking two or more inhibitors (of the same type or of different types). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Another possibility for cross-linking takes advantage of the PI linkage signal sequence in PI-linked LFA-3, or fragments thereof. Specifically, DNA encoding the PI-linkage signal sequence (e.g., amino acids 162-212 of SEQ ID NO:4) is ligated downstream of DNA encoding a desired polypeptide, preferably a soluble LFA-3 polypeptide. If this construct is expressed in an appropriate eukaryotic cell, the cell will recognize the PI linkage signal sequence and will covalently link PI to the polypeptide. The hydrophobic property of the PI may then be exploited to form micellar aggregates of the polypeptides.

Also useful are inhibitors linked to one or more cytotoxic or pharmaceutical agents. Useful pharmaceutical agents include biologically active peptides, polypeptides and proteins, such as antibody homologs specific for a human polypeptide other than CD2 or LFA-3, or portions thereof. Useful pharmaceutical agents and cytotoxic agents also include UV radiation (e.g., UVB), cyclosporin A, prednisone, FK506, methotrexate, steroids, retinoids, interferon, and nitrogen mustard.

Preferred inhibitors derivatized with a pharmaceutical agent include recombinantly-produced polypeptides in which a soluble LFA-3 polypeptide, soluble CD2 polypeptide, or a peptidyl CD2 or peptidyl LFA-3 mimetic agent is fused to all or part of an immunoglobulin heavy chain hinge region and all or part of a heavy chain constant region. Preferred polypeptides for preparing such fusion proteins are soluble LFA-3 polypeptides Most preferred are fusion proteins containing amino acid 1-92 of mature LFA-3 fused to a portion of a human IgG₁ hinge region (including the C-terminal ten amino acids of the hinge region containing two cysteine residues thought to participate in interchain disulfide bonding) and the C_{H}2 and C_{H}3 regions of an IgG₁ heavy chain constant domain. Such fusion proteins are expected to exhibit prolonged serum half-lives and enable inhibitor dimerization.

The utility in the methods of this invention of specific soluble CD2 polypeptides, soluble LFA-3 polypeptides, anti-LFA-3 antibody homo logs, anti-CD2 antibody homologs or CD2 and LFA-3 mimetic agents may easily be determined by assaying their ability to inhibit the LFA-3/CD2 interaction. This ability may be assayed, for example, using a simple cell binding assay that permits visual (under magnification) evaluation of the ability of the putative inhibitor to inhibit the interaction between LFA-3 and CD2 on cells bearing these molecules. Jurkat cells are preferred as the CD2⁺ substrate and sheep red blood cells or human JY cells are preferred as the LFA-3⁺ substrate. The binding characteristics of soluble polypeptides, antibody homologs and mimetic agents useful in this invention may be assayed in several known ways, such as by radiolabeling the antibody homolog, polypeptide or agent (e.g., with ³⁵S or ¹²⁵I) and then contacting the labeled polypeptide, mimetic agent or antibody homolog with CD2⁺ of LFA-3⁺ cells, as appropriate. Binding characteristics may also be assayed using an appropriate enzymatically labelled secondary antibody. Rosetting competition assays such as those described by Seed et al. (Proc. Natl. Acad. Sci. USA, 84, pp. 3365-69 (1987)) may also be used.

Combination Therapy

The agents, e.g., soluble CD2 binding LFA-3 polypeptides, may be used in combination with other therapies, e.g., other agents. The other agent(s) are referred to herein as “second agent(s)” or “additional agents” and include one or more of: an immunosuppressant (e.g., methotrexate, cyclosphorin, or chlorambucil), cyclophosphamide, prednisone, FK506, steroids, retinoids, interferon, nitrogen mustard, a cytokine binding agent (e.g., type 2 cytokine binding agent, e.g., an IL-2- or IL-8-binding agent, e.g., an anti-IL-2 or IL-8 monoclonal antibody (Abgenix)), an inhibitor of an ICAM/LFA-1 interaction, e.g., an ICAM-binding agent (e.g., an antibody, e.g., a monoclonal antibody) against ICAM-1 (e.g., a humanized, chimeric, or human anti-ICAM-1 antibody); or an LFA-1 (also known as CD11a) binding agent (e.g., an antibody, e.g., a monoclonal antibody) against LFA-1 (e.g., a humanized, chimeric, or human anti-LFA-1 antibody, e.g., Raptiva (Genentech/Xoma)); a costimulatory molecule binding agent, e.g., a B7-1 (CD80) binding agent (e.g., an anti-B7-1 monoclonal antibody (IDEC); a vasolidator (e.g., an ACE inhibitor or minoxidil); a corticosteroid or penicillamine. In one embodiment, the agent, e.g., an inhibitor of the CD2:LFA-3 interaction, is administered in combination with one or more inhibitors of interleukin-1 (IL-1), IL-2, IL-4, IL-6, IL-8, TGF-β, PDGF, granzyme A or leukotriene B4. Such combination therapy may advantageously utilize lower dosages of the therapeutic or prophylactic agents.

Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated. In some embodiments, the delivery of one treatment is still occurring when the delivery of the second begins, so that there is overlap. This is sometimes referred to herein as “simultaneous” or “concurrent delivery.” In other embodiments, the delivery of one treatment ends before the delivery of the other treatment begins. In some embodiments of either case, the treatment is more effective because of combined administration. E.g., the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder, e.g., reduction in T cell level or activity, is greater than what would be observed with one treatment delivered in the absence of the other. The effect of the two treatments can be partially additive, wholly additive, or greater than additive. The delivery can be such that an effect of the first treatment delivered is still detectable when the second is delivered, e.g., when the CD2- or LFA-3 binding agent is delivered first, a reduction in T cell level or activity is still detectable when second agent is delivered. In a preferred embodiment, a delivery of the first treatment and a delivery of the second treatment occur within 1, 2, 5, 10, 15, or 30 days of one another.

In a preferred embodiment, the CD2-binding agent (e.g., LFA-3/Ig fusion), the second agent (or both) or a pharmaceutical composition containing the same is administered systemically, e.g., intravenously, intramuscularly, subcutaneously, intra-articularly, transdermally, intrathecally, periostally, intratumorally, intralesionally, perilesionally by infusion (e.g., using an infusion device), orally, topically or by inhalation. Preferably, the CD2-binding agent is administered intramuscularly or intravenously. In other embodiment, the CD2-binding agent is administered locally, e.g., topically or by needleless injection, to an affected area.

The parenteral administration of the CD2-binding agent (e.g., LFA-3/Ig fusion), the second agent (or both) or a pharmaceutical composition containing the same can be effected using a needle or a needleless syringe by procedures known in the art. Examples of needleless syringe systems and modes of administration are described in U.S. Pat. No. 6,132,395, U.S. Pat. No. 6,096,002, U.S. Pat. No. 5,993,412, U.S. Pat. No. 5,893,397, U.S. Pat. No. 5,520,639, U.S. Pat. No. 5,503,627, U.S. Pat. No. 5,399,163, U.S. Pat. No. 5,383,851, U.S. Pat. No. 5,312,577, U.S. Pat. No. 5,312,335, the contents of all of which are hereby incorporated by reference.

Pharmaceutical Compositions

Preferably, an effective amount of the CD2:LFA3 inhibitor (e.g., a soluble, CD2-binding LFA-3 polypeptide described herein) is administered. By “effective amount” is meant an amount capable of lessening the spread or severity of the conditions described herein. In therapeutic embodiments, an effective amount of the agent refers to an amount of an agent that is effective, upon single or multiple dose administration to the subject, at inhibiting, reducing, or ameliorating the disorder (e.g., improving the PASI score or PGA score for a psoriasis patient), or in prolonging the survival of the patient with the disorder beyond that expected in the absence of such treatment. The amelioration of psoriasis, for example, is predicted to lead to improved quality of life, as assessed, e.g., by the SF-36 health questionnaire developed by RAND Health, a division of the RAND Corporation (Santa Monica, Calif.). An effective amount does not necessarily indicate a total elimination of the disorder. In prophylactic embodiments, an effective amount of a CD2- or LFA-3 binding agent described herein refers to an amount of an agent that is effective, upon single- or multiple-dose administration to the subject, in preventing or delaying the occurrence of the onset or recurrence of the disorder.

It will be apparent to those of skill in the art that the effective amount of agent will depend, inter alia, upon the disorder treated (e.g., T cell mediated disorder of the skin vs. T cell mediated disorder of an organ other than the skin), administration schedule, the unit dose administered, whether the agent is administered in combination with other therapeutic agents, the immune status and health of the patient, the therapeutic or prophylactic activity of the particular agent administered and the serum half-life. Depending on the disorder to be treated the agent may be packaged differently.

Preferably, a soluble, CD2-binding LFA-3 polypeptide (e.g., LFA3TIP) is administered at a dose between about 0.001 and about 50 mg of the agent per kg body weight, more preferably, between about 0.01 and about 10 mg of the agent per kg body weight, most preferably between about 0.1 and about 4 mg of the agent per kg body weight. In preferred embodiment, the soluble, CD2-binding LFA-3 polypeptide is administered at a unit dosage ranging from 2 to 15 mg when administered by IV route (for example, 7.5 mg IV bolus) and a dosage ranging from 2 to 30 mg when administered by IM route (for example, 10 mg or 15 mg IM injection). IM and IV administration are preferred.

Unit doses are typically administered until an effect is observed. The effect may be measured by a variety of methods, including in vitro T cell activity assays and clearing or improvement of affected skin areas, or improvement in other affected body areas as may be relevant to the particular disorder. Preferably, the unit dose is administered at regular intervals during a treatment cycle, such as once a week. More preferably, it is administered at regular intervals, e.g., at weekly intervals for an administration period of several weeks, e.g., twelve weeks. More frequent administrations, e.g., two or three times per week are also envisioned and may be adapted if the subject's disorder is severe or if urgent intervention is indicated. Less frequent administrations, e.g., once or twice per month, are also envisioned and may be adopted if the subject responds well to therapy such that maintenance dosing is appropriate. It will be recognized, however, that lower or higher dosages and other administration schedules may be employed during any one particular cycle of administration.

The agent, e.g., CD2-binding LFA-3 polypeptide (e.g., AMEVIVE) is also preferably administered in a composition including a pharmaceutically acceptable carrier. By “pharmaceutically acceptable carrier” is meant a carrier that does not cause an allergic reaction or other untoward effect in patients to whom it is administered.

Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the agent.

Formulations, e.g., pharmaceutical formulations, of the CD2-binding agent(s) can be prepared in aqueous or non-aqueous, e.g., lyophilized, forms. Preferred pharmaceutical formulations are suitable for injection. An example of an aqueous formulation encompassed by the present invention includes phosphate buffered saline (PBS) frozen liquid formulation. An example of a lyophilized formulation includes one or more of: citrate, glycine and sucrose. For example, a preferred lyophilized formulation includes 1 to 5% sucrose, preferably 2.5% sucrose, and 0.5% to 2% glycine, preferably 1% glycine, in sodium citrate-citric buffer (at least 10 mM, preferably 25 mM) buffered to a pH of at least about 4, preferably, 5, more preferably 6 (or even more preferably, 6.8).

The second agent may be administered in a single dosage form with the CD2-binding agent(s) (i.e., as part of the same pharmaceutical composition), a multiple dosage form, separately from the CD2-binding agent(s) but concurrently, or a multiple dosage form wherein the two components are administered separately and sequentially. Alternatively, the CD2-binding agent and the other active agent may be in the form of a single conjugated molecule. Conjugation of the two components may be achieved by standard cross-linking techniques well known in the art. A single molecule may also take the form of a recombinant fusion protein. In addition, a pharmaceutical composition useful in the present invention may be used in combination with other therapies such as PUVA, chemotherapy and UV light. Such combination therapies may advantageously utilize lower dosages of the therapeutic or prophylactic agents.

The CD2-binding agent, or pharmaceutical composition, may be in a variety of forms. These include, for example, solid, semi-solid and liquid dosage forms, such as tablets, pills, powders, liquid solutions, dispersions or suspensions, liposomes, suppositories, injectable, infusible, and topical preparations. The preferred form depends on the intended mode of administration and therapeutic application. The preferred forms are injectable or infusible solutions.

The invention includes formulations suitable for use as topically applied sunscreens or UV-protectants. Preferred embodiments include AMEVIVE preparations. The active ingredient can be formulated in a liposome. The product can be applied before, during, or after UV exposure, or before, during, or after the development of redness.

Sequences

The following is a summary of the sequences described in U.S. Pat. No. 6,162,432 and referred to throughout the application:

| | |
|---|---|
| SEQ ID NO: 1 | DNA sequence of transmembrane LFA-3 |
| SEQ ID NO: 2 | Amino acid sequence of transmembrane LFA-3 |
| SEQ ID NO: 3 | DNA sequence of PI-linked LFA-3 |
| SEQ ID NO: 4 | Amino acid sequence of PI-linked LFA-3 |
| SEQ ID NO: 5 | DNA sequence of CD2 |
| SEQ ID NO: 6 | Amino acid sequence of CD2 |
| SEQ ID NO: 7 | DNA sequence of AMEVIVE |
| SEQ ID NO: 8 | Amino acid sequence of AMEVIVE |

### EXAMPLES

### Example 1

### Multi-Course Treatment of Psoriasis with AMEVIVE® (Alefacept)

This example examined efficacy and safety in patients who have received a multiple course of treatment of up to nine cycles of therapy with AMEVIVE® over 4.5 years.

Treatment

The initial treatment cycle in the open-label study is referred to as cycle A. Subsequent cycles are labeled cycle B, C, D, and so on. Patients received once weekly treatment for 12 weeks (administration period) followed by 12 weeks of observation (rest period) in each cycle.

AMEVIVE 7.5 mg was administered by intravenous (IV) bolus injection.

Prior to initiation of treatment in the extension study the need for systemic therapy was established based on physician's assessment of disease severity, and CD4+ T-cell counts were at or above the lower limit of normal (LLN; 404 cells/mm³).

Eligibility for a subsequent cycle was based on the aforementioned criteria and, in addition: patients must have received ≧8 doses of AMEVIVE during the 12-week treatment period of the previous cycle and for cycle C and subsequent cycles, lymphocyte counts were required to be ≧75% of the count recorded at the screening visit of the study.

Within any given cycle, if patients had CD4+ T-cell counts <300 but >200 cells/mm³, the dose of AMEVIVE was reduced by 50% (3.75 mg). If CD4+ T-cell counts were <200 cells/mm³, the scheduled dose was withheld. If CD4+ T-cell counts were <200 cells/mm³ for 4 consecutive visits, AMEVIVE was permanently withheld. The AMEVIVE dose was withheld for 2 weeks when evidence of a clinically significant infection was seen.

Assessment

Efficacy was assessed by the Psoriasis Area and Severity Index (PASI) and by Physician Global Assessment (PGA). For cycle A, assessments were made at weeks 1, 3, 5, 7, 9, 11, and 12 during treatment and at 2, 4, 6, 8, and 12 weeks after treatment. For subsequent cycles, assessments were made at weeks 1 and 7 during treatment and at 2 and 12 weeks after treatment. The proportions of patients who achieved ≧50% and ≧75% improvement in PASI from baseline (PASI 50 and PASI 75, respectively) and those who achieved PGA of “clear” or “almost clear” were reported.

Total lymphocyte and lymphocyte subset analyses were conducted at each study visit, except at 4 weeks after treatment in cycle A and at 4 and 8 weeks after treatment in all subsequent cycles. Patients with new or ongoing viral, bacterial, or fungal infections were monitored at all visits. Adverse events were monitored throughout the study.

Results

At the time of this analysis, patients have received repeated cycles of therapy with AMEVIVE over 4.5 years. In this study, 175 patients have received ≧1 cycle of AMEVIVE; 126 received ≧2 cycles; 96 received ≧3 cycles; and 71 received ≧4 cycles. Some patients have received up to 9 cycles of AMEVIVE as a result of exposure to AMEVIVE in prior phase 2 studies.

Efficacy

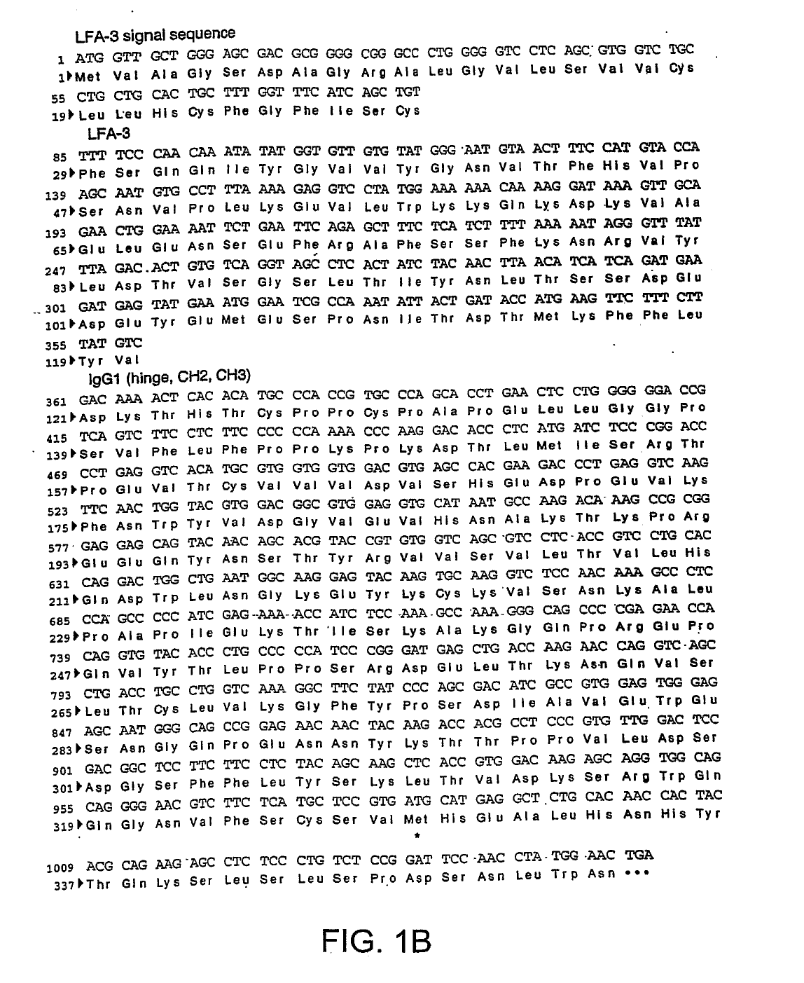

The proportions of patients achieving PASI 50 at 2 or 12 weeks after treatment for cycles A-D are shown in FIG. 2. The response rate of cycles C and D is notably increased compared to cycles A and B. The proportions of patients achieving PASI 75 were 29%, 33%, 34%, and 52% in cycles A, B, C, and D, respectively. The corresponding response rates for a PGA of “clear” or “almost clear” were 24%, 29%, 33%, and 34%, respectively.

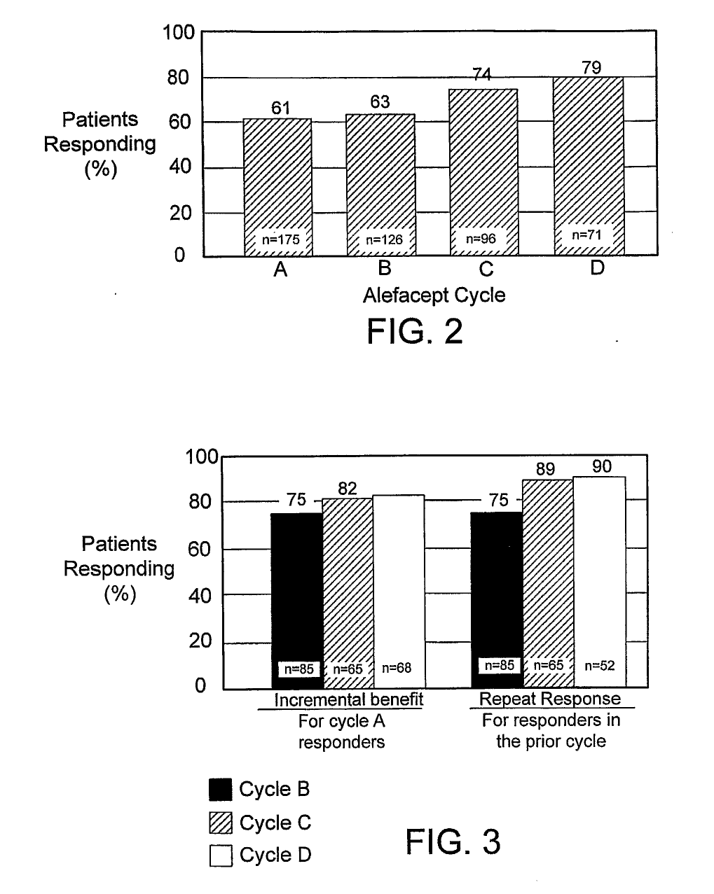

For cycles A-D, the incremental benefit and repeat response of additional cycles of therapy with AMEVIVE are shown in FIG. 3. For cycle A responders (i.e., patients who achieved PASI 50 in cycle A) who received additional cycles of AMEVIVE, the proportions of patients who achieved PASI 50 increased with each subsequent cycle. In general, patients continued to respond to repeat treatment with AMEVIVE with no evidence of tachyphylaxis. Of those who achieved PASI 50 in a given cycle, 75% to 90% of patients achieve PASI 50 in subsequent cycles (i.e., repeat response).

Adverse Events

The incidence of adverse events, in general, did not vary considerably across the cycles. The overall safety profile of AMEVIVE following administration of multiple cycles (a multiple course of treatment) is similar to that reported in phase 3 studies. The incidence of serious adverse events was 7% or less in any cycle, and the spectrum of serious adverse events was similar to previous phase 2 and 3 studies.

Two patients each in cycles A and B and 1 patient in cycle E discontinued treatment because of an adverse event. The incidence of malignancies was low: 3% or less in any cycle; the majority were skin cancers.

Duration of Treatment-Free Response

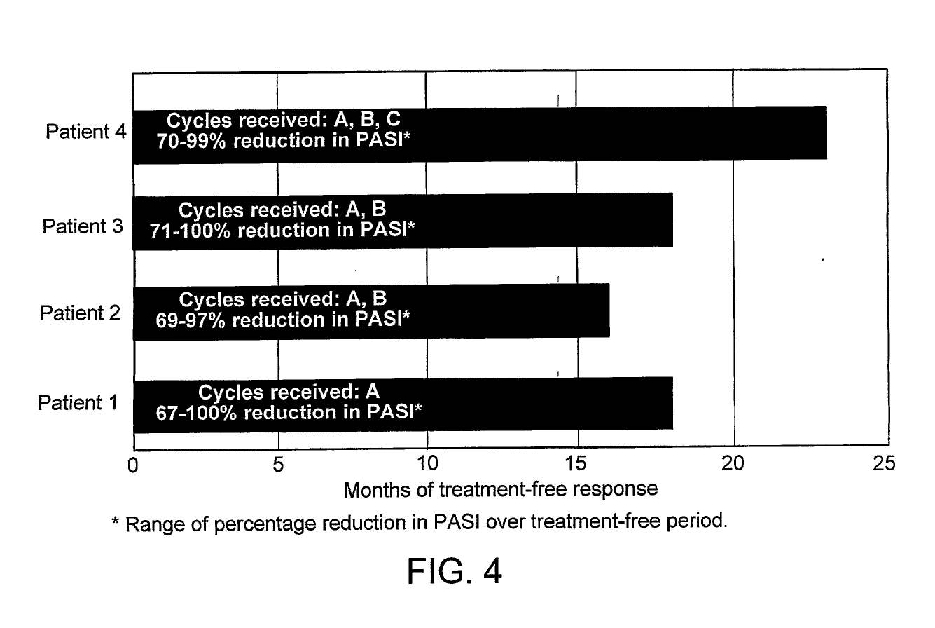

In the phase 3 studies, the remittive action of AMEVIVE was demonstrated, with patients maintaining PASI 50 responses for a median of 7 months. In this example, some patients have been followed for prolonged periods after successful treatment cycles. FIG. 4 shows the maximum length of response time in 4 such patients. The response to therapy with AMEVIVE has been maintained for 18-24 months in some patients.

Lymphocyte Counts

Across multiple cycles of treatment with AMEVIVE, decreases in lymphocyte counts were consistent. The decreases in lymphocyte counts observed with each cycle were not cumulative.

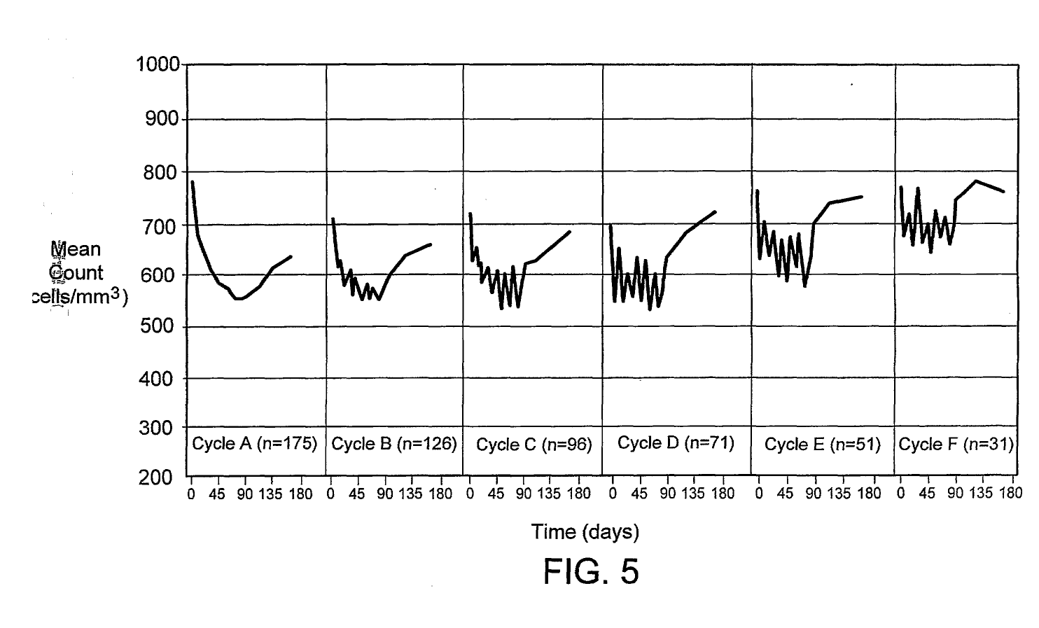

Mean CD4+ T-cell counts remained above the LLN for all cycles and did not decrease with multiple-course exposure to AMEVIVE (FIG. 5).

In sum, this study shows that a multiple course of treatment (3 cycles of treatment or more) provides more significant results than a single course of therapy, with no apparent additional risk of side effects. Multiple cycles of AMEVIVE were well-tolerated by patients, and the incidence of adverse events did not vary considerably across the cycles.

### Example 2

### Multi-course treatment of psoriasis with AMEVIVE

This example examined the clinical response to a second cycle of treatment with AMEVIVE in psoriasis patients who failed to achieve a ≧50% reduction in Psoriasis Area and Severity Index (PASI 50) or a ≧25% reduction (PASI 25) during a first cycle of treatment with AMEVIVE, as well as the efficacy of multiple cycles of therapy.

Patients

Patients were ≧16 years old and had chronic plaque psoriasis for ≧12 months that involved ≧10% of body surface area. CD4+ T-cell counts were required to be above the lower limit of normal (LLN). Treatment with phototherapy, systemic retinoids, systemic corticosteroids, systemic fumarates, immunosuppressants (methotrexate, cyclosporine, azathioprine, and thioguanine), and high-potency topical corticosteroids was prohibited within 4 weeks before treatment with AMEVIVE and throughout the studies. Use of moderate-potency topical corticosteroids, topical retinoids, coal tar, keratolytics, and vitamin D analogues was prohibited within 2 weeks of treatment with AMEVIVE and throughout the studies, except on the scalp, palms, groin, and soles. To be eligible for enrollment in the extension studies, patients were required to have received ≧8 doses of AMEVIVE and completed the final follow-up visit of the previous cycle of therapy. Patients were excluded from the extension studies if they enrolled in any other investigational studies of drug or nondrug therapy or initiated alternative systemic psoriasis treatments, phototherapy, or other disallowed therapies prior to week 8 of the previous cycle.

Treatment

The phase 3 studies were multicenter, randomized, double-blind, and placebo-controlled (Krueger et al., J. Am. Acad. Dermatol. 47:821-833, 2002; Lebwohl et al., Arch. Dermatol. 139:719-727, 2003). In the phase 3 study of intravenous (IV) treatment with AMEVIVE, patients received two cycles of treatment, where each cycle consisted of (i) a 12 week administration period of either once weekly AMEVIVE (7.5 mg) or placebo, and (ii) a 12 week follow-up (rest period). In the phase 3 study of intramuscular (IM) treatment with AMEVIVE, patients received a single cycle of treatment that consisted of a 12 week administration period of either AMEVIVE (10 mg or 15 mg) or placebo once weekly, followed by 12 weeks of observation (rest period).

In the multiple course therapy studies, patients who received additional cycles of treatment with AMEVIVE (same dosage regimen) were those whose disease had progressed to require systemic therapy or phototherapy, as determined by the investigator, and had circulating CD4+ T-cell counts at or above the LLN (Gordon et al., J. Drugs Dermatol. 2:624-628, 2003).

Assessment

During the phase 3 studies, PASI and Physician Global Assessment (PGA) were evaluated at baseline, every 2 weeks during treatment, and every 2 to 4 weeks during follow-up. PASI and PGA were also assessed at several time points during the IV multiple course study, whereas only PGA was assessed during the IM multiple course study.

Analyses

Data from the 2-cycle phase 3 study of IV treatment with AMEVIVE were used to determine the efficacy of a second cycle of treatment with AMEVIVE in two groups of patients: (a) those who failed to achieve PASI 50 in the first cycle and (b) those who failed to achieve PASI 25 in the first cycle. Proportions of patients who failed to achieve PASI 50 and PASI 25 during cycle 1 of treatment with AMEVIVE who achieved PASI 50 or PASI reductions of ≧75% (PASI 75) at any time during a second cycle of treatment were determined. Odds ratios and corresponding 95% confidence intervals (CIs) were calculated to compare response rates between patients who received a second cycle of treatment with AMEVIVE and those who received placebo. Proportions of patients who achieved PASI 50, PASI 75, and PGA of “clear” or “almost clear” at any time during each of the IV treatment cycles, and the proportion of patients who achieved a PGA of “clear” or “almost clear” at any time during each of the IM treatment cycles were also determined.

Results

Patients had a mean age of 45 years, and 70% were male. The median duration of psoriasis was 19 years, and the median body surface area involvement was 22%. The number of patients receiving each cycle of IV and IM treatment with AMEVIVE is summarized in Table 1.

TABLE 1
Number of patients receiving multiple cycles of AMEVIVE.

| | Treatment Cycle | | | | |
|---|---|---|---|---|---|
| | 1 | 2 | 3 | 4 | 5 |
| IV AMEVIVE | 521 | 327 | 217 | 158 | 39 |
| IM AMEVIVE | 457 | 320 | 156 | 100 | 50 |

Efficacy of a Second Cycle of Treatment in Patients who Previously Failed to Achieve PASI 50 or PASI 75

PASI improvement was observed during a second cycle of IV treatment with AMEVIVE in 93% of patients who failed to achieve PASI 50 during the first cycle and 89% of patients who failed to achieve PASI 25 during the first cycle. Treatment with AMEVIVE in the second cycle resulted in significantly greater proportions of patients achieving PASI 50 and PASI 75 than placebo treatment (Table 2). 19% of patients who failed to achieve PASI 50 during the initial cycle of treatment with AMEVIVE achieved PASI 75 during the second cycle and 53% achieved PASI 50.14% of patients who failed to achieve PASI 25 during the initial cycle of treatment with AMEVIVE achieved PASI 75 during the second cycle and 47% achieved PASI 50 (Table 2). Odds ratios showed that patients who failed to achieve PASI 50 or PASI 25 in cycle 1 were 2 to 3 times more likely to achieve PASI 50 or PASI 75 in cycle 2 compared with patients who did not receive another cycle (Table 2).

TABLE 2
PASI improvement at any time during a second cycle of AMEVIVE
in patients who failed to achieve PASI 50 or PASI 25
during the first cycle.
Treatment

| | | |
|---|---|---|
| | AMEVIVE- | Odds ratio |
| AMEVIVE—AMEVIVE | Placebo | (95% CI) |

Patients who did not achieve
PASI 50 in first AMEVIVE cycle

| | | | |
|---|---|---|---|
| N | 97 | 86 | |
| PASI 75, | 18 (18.6) | 7 (8.1) | 2.57 (1.02-6.50) |
| n (%) | | | |
| PASI 50, | 51 (52.6) | 28 (32.6) | 2.30 (1.26-4.19) |
| n (%) | | | |
| Any PASI | 90 (92.8) | 74 (86.0) | 2.08 (0.78-5.56) |
| improve- | | | |
| ment, n (%) | | | |

Patients who did not achieve
PASI 25 in first AMEVIVE cycle

| | | | |
|---|---|---|---|
| N | 66 | 46 | |
| PASI 75, | 9 (13.6) | 3 (6.5) | 2.26 (0.58-8.86) |
| n (%) | | | |
| PASI 50, | 31 (47.0) | 11 (23.9) | 2.82 (1.23-6.48) |
| n (%) | | | |
| Any PASI | 59 (89.4) | 38 (82.6) | 1.77 (0.59-5.29) |
| improve- | | | |
| ment, n (%) | | | |

Efficacy of Multiple Course Therapy

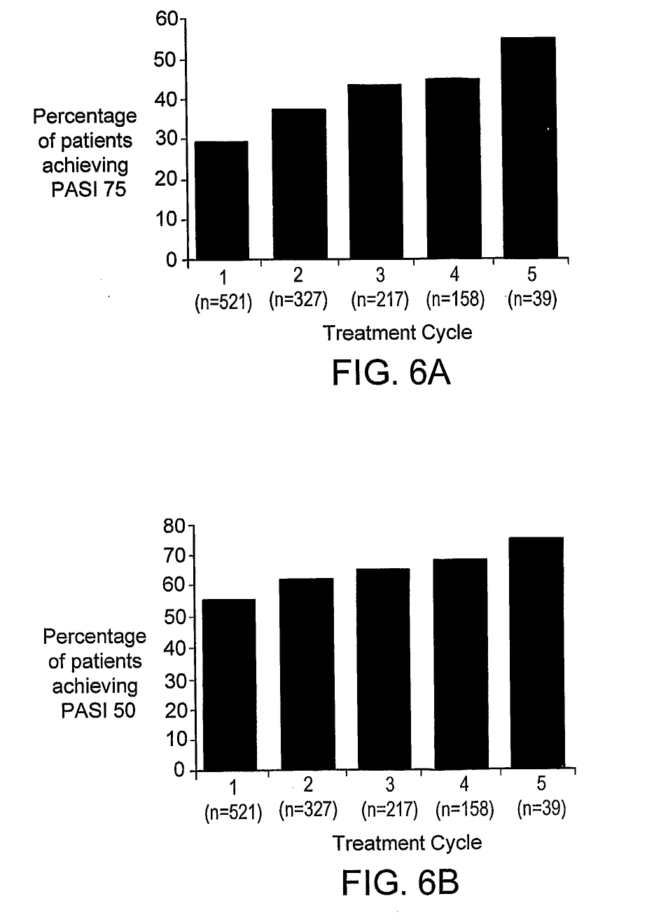

Patients who received multiple cycles of IV treatment with AMEVIVE showed incremental improvement in PASI during each subsequent treatment cycle. The proportion of patients achieving PASI 75 increased from 29% during cycle 1 to a maximum of 54% during cycle 5 (FIG. 6A). Similarly, the proportion of patients achieving PASI 50 increased from 56% during cycle 1 to a maximum of 74% during cycle 5 (FIG. 6B).

PGA results further support the incremental clinical improvement in psoriasis observed with multiple cycles of treatment with AMEVIVE. 23% of patients had a PGA of “clear” or “almost clear” during cycle 1 of IV treatment with AMEVIVE.

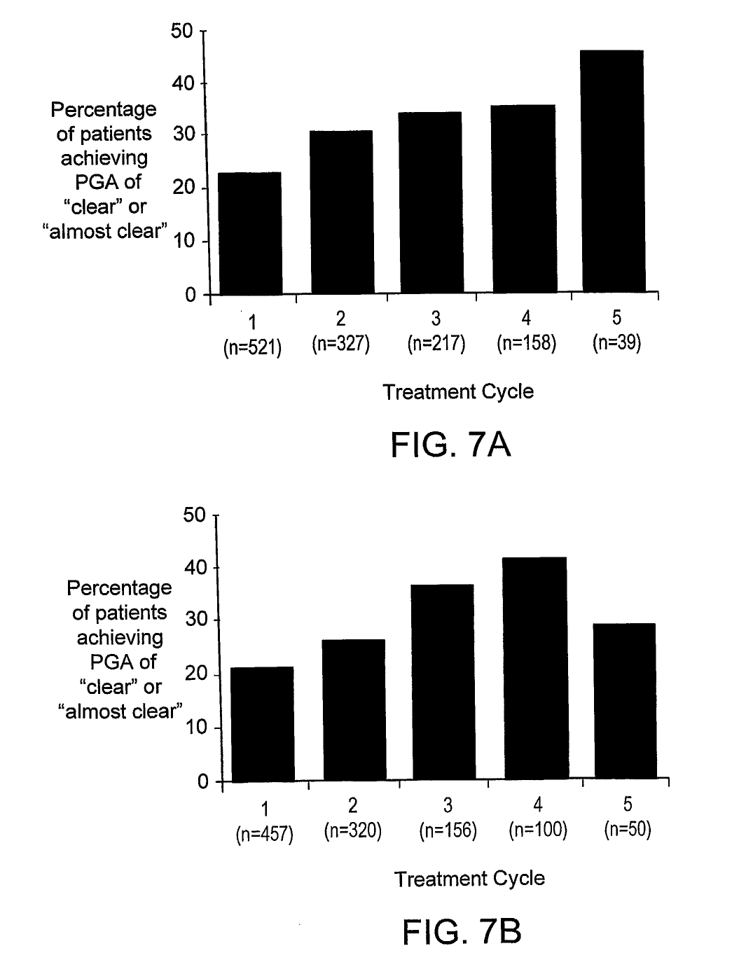

During cycle 5, 44% of patients achieved this level of response (FIG. 7A). For IM treatment with AMEVIVE, PGA “clear”/“almost clear” response rates increased from 21% during cycle 1 to a maximum of 41% during cycle 4 (FIG. 7B).

In sum, incremental clinical improvement is seen following successive cycles of treatment with AMEVIVE, indicating its efficacy for the long-term treatment of patients with chronic plaque psoriasis. Multiple course therapy data show that patient response to additional treatment with AMEVIVE is incrementally beneficial regardless of response to the initial treatment.

1. A method of treating a subject who has psoriasis, the method comprising administering a multiple course of treatment of a soluble CD2-binding LFA-3 polypeptide to the subject, wherein the multiple course comprises multiple cycles of treatment, and wherein each cycle comprises an administration period and a rest period.

2. The method of claim 1, wherein the soluble CD2-binding LFA-3 polypeptide is an LFA-3 fusion protein.

3. The method of claim 1, wherein the soluble CD2-binding LFA-3 polypeptide is an LFA-3/immunoglobulin (Ig) fusion protein.

4. The method of claim 1, wherein the soluble CD2-binding LFA-3 polypeptide comprises a soluble LFA-3 polypeptide fused to all or part of an Ig heavy chain hinge region and all or part of a heavy chain constant region.

5. The method of claim 1, wherein the soluble CD2-binding LFA-3 polypeptide comprises a fusion protein consisting of the amino terminal 92 amino acids of mature LFA-3, the C-terminal 10 amino acids of a human IgG1 hinge region, a CH2 region of a human IgG1 heavy chain, and at least part of a CH3 region of a human IgG1 heavy chain.

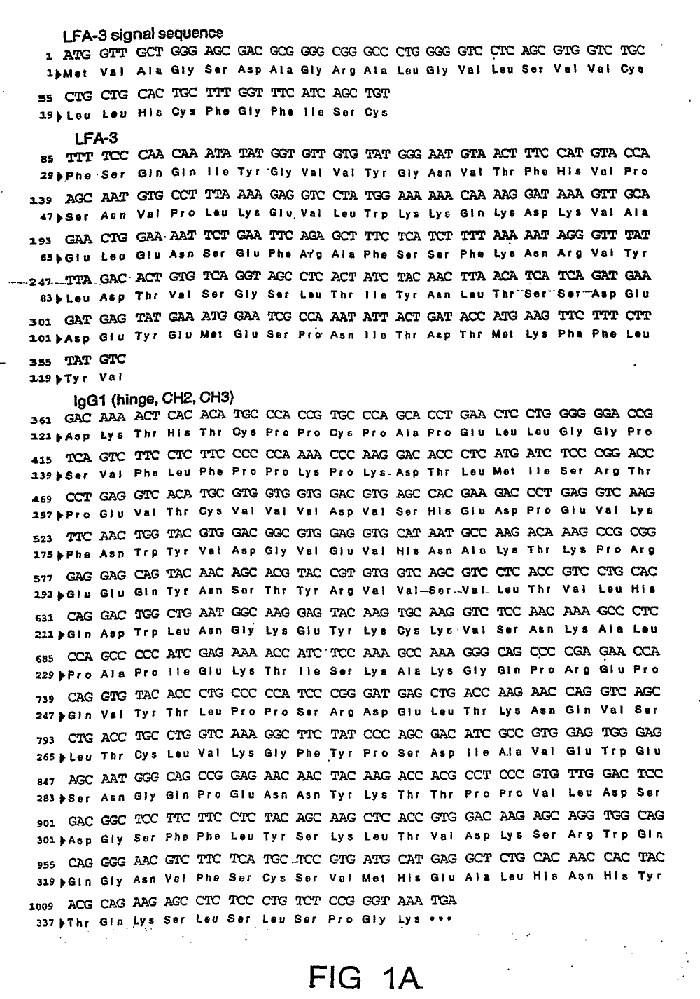

6. The method of claim 1, wherein the soluble CD2-binding LFA-3 polypeptide is AMEVIVE (FIG. 1).

7. The method of claim 1, wherein the soluble CD2-binding LFA-3 polypeptide is encoded by an insert contained in plasmid pSAB152, deposited with American Type Culture Collection under the accession number ATCC 68720.

8. The method of claim 1, wherein the multiple course comprises at least four cycles of treatment.

9. The method of claim 1, wherein the multiple course comprises at least five cycles of treatment.

10. The method of claim 1, wherein the multiple course comprises at least six cycles of treatment.

11. The method of claim 1, wherein the multiple course comprises at least seven cycles of treatment.

12. The method of claim 1, wherein the multiple course comprises at least eight cycles of treatment.

13. The method of claim 1, wherein the rest period of each successive cycle of the multiple course is longer than the rest period of a previous cycle in the multiple course.

14. The method of claim 1, wherein the rest period of the last cycle of the multiple course is at least 2 years.

15. The method of claim 1, wherein the rest period of the last cycle of the multiple course is at least 3 years.

16. The method of claim 1, wherein the administration period of each cycle of the multiple course is at least 8 weeks.

17. The method of claim 1, wherein the administration period of each cycle of the multiple course is at least 10 weeks.

18. The method of claim 1, wherein the administration period of each cycle of the multiple course is at least 12 weeks.

19. The method of claim 1, wherein the polypeptide is administered intramuscularly.

20. The method of claim 1, wherein the polypeptide is administered intravenously.

21. The method of claim 1, wherein the polypeptide is administered at a unit dosage ranging from 2 to 30 mg.

22. The method of claim 1, wherein the method further comprises administering to the subject an additional therapeutic or prophylactic agent during the multiple course of treatment.

23. A method of treating a subject in need of treatment for psoriasis, the method comprising administering a multiple course of treatment of AMEVIVE (FIG. 1) to the subject, wherein the multiple course of treatment comprises at least three cycles of treatment, each cycle of treatment comprising an administration period of once-weekly administration of AMEVIVE (FIG. 1) for 12 weeks, followed by a rest period of at least 12 weeks.

24. The method of claim 23, wherein the multiple course of treatment comprises at least four cycles of treatment.

25. The method of claim 23, wherein the multiple course of treatment comprises at least five cycles of treatment.

26. The method of claim 23, wherein the method comprises evaluating the subject for the effects of AMEVIVE (FIG. 1) during one or both of the administration period and the rest period of each cycle in the multiple course.

27. The method of claim 23, wherein the method further comprises administering to the subject an additional therapeutic or prophylactic agent during the multiple course of treatment.

28. A method of treating a subject having psoriasis, the method comprising (a) selecting a subject on the basis of having had at least two cycles of treatment with a soluble CD2-binding LFA-3 polypeptide and (b) administering a third cycle of treatment of a soluble CD2-binding LFA-3 polypeptide to the subject.

29. The method of claim 28, wherein the soluble CD2-binding LFA-3 polypeptide is AMEVIVE (FIG. 1).

30. A kit comprising a pharmaceutical composition comprising AMEVIVE and instructions to administer the pharmaceutical composition to a patient who has previously had two cycles of treatment with AMEVIVE (FIG. 1).


SEQUENCE LISTING (8 sequences)

SEQ ID NO: 1 - DNA, 753 bp, Homo sapiens
  CDS (1)..(750)
  sig_peptide (1)..(84)
  misc_feature (1)..(750): “Human transmembrane LFA-3”
  mat_peptide (85)..(750)
  misc_feature (646)..(646): “Transmembrane domain”
atggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgaattcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagcagcggtcattcaagacacagatatgcacttatacccataccattagcagtaattacaacatgtattgtgctgtatatgaatggtattctgaaatgtgacagaaaaccagacagaaccaactccaattga

SEQ ID NO: 2 - protein, 250 aa, Homo sapiens
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRYALIPIPLAVITTCIVLYMNGILKCDRKPDRTNSN

SEQ ID NO: 3 - DNA, 723 bp, Homo sapiens
  CDS (1)..(720)
  sig_peptide (1)..(84)
  misc_feature (1)..(720): “Human PI-linked LFA-3”
  mat_peptide (85)..(720)
  misc_feature (568)..(720): “signal sequence for PI-linkage”
atggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgaattcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagcagcggtcattcaagacacagatatgcacttatacccataccattagcagtaattacaacatgtattgtgctgtatatgaatggtatgtatgctttttaa

SEQ ID NO: 4 - protein, 240 aa, Homo sapiens
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRYALIPIPLAVITTCIVLYMNGMYAF

SEQ ID NO: 5 - DNA, 1056 bp, Homo sapiens
  CDS (1)..(1053)
  sig_peptide (1)..(72)
  misc_feature (1)..(1053): “Human CD2”
  mat_peptide (73)..(1053)
  misc_feature (628)..(702): “Transmembrane domain”
atgagctttccatgtaaatttgtagccagcttccttctgattttcaatgtttcttccaaaggtgcagtctccaaagagattacgaatgccttggaaacctggggtgccttgggtcaggacatcaacttggacattcctagttttcaaatgagtgatgatattgacgatataaaatgggaaaaaacttcagacaagaaaaagattgcacaattcagaaaagagaaagagactttcaaggaaaaagatacatataagctatttaaaaatggaactctgaaaattaagcatctgaagaccgatgatcaggatatctacaaggtatcaatatatgatacaaaaggaaaaaatgtgttggaaaaaatatttgatttgaagattcaagagagggtctcaaaaccaaagatctcctggacttgtatcaacacaaccctgacctgtgaggtaatgaatggaactgaccccgaattaaacctgtatcaagatgggaaacatctaaaactttctcagagggtcatcacacacaagtggaccaccagcctgagtgcaaaattcaagtgcacagcagggaacaaagtcagcaaggaatccagtgtcgagcctgtcagctgtccagagaaaggtctggacatctatctcatcattggcatatgtggaggaggcagcctcttgatggtctttgtggcactgctcgttttctatatcaccaaaaggaaaaaacagaggagtcggagaaatgatgaggagctggagacaagagcccacagagtagctactgaagaaaggggccggaagccccaccaaattccagcttcaacccctcagaatccagcaacttcccaacatcctcctccaccacctggtcatcgttcccaggcacctagtcatcgtcccccgcctcctggacaccgtgttcagcaccagcctcagaagaggcctcctgctccgtcgggcacacaagttcaccagcagaaaggcccgcccctccccagacctcgagttcagccaaaacctccccatggggcagcagaaaactcattgtccccttcctctaattaa

SEQ ID NO: 6 - protein, 351 aa, Homo sapiens
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 7 - DNA, 1050 bp, Homo sapiens
  CDS (1)..(1041)
  sig_peptide (1)..(84)
  mat_peptide (85)..(1041)
  misc_feature (85)..(1041): “LFA3TIP”
  misc_feature (360)..(361): “LFA-3/IgG fusion point”
atggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgaattcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgg

SEQ ID NO: 8 - protein, 347 aa, Homo sapiens
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK